(19) United States
(12) Patent Application Publication
(10) Pub. No.: US 2010/0177867 A1
(43) Pub. Date: Jul. 15, 2010

(54) GANTRY X-RAY TRANSMISSIVE ELEMENT

(75) Inventors: Peter J. Kozelj, Willoughby Hills, OH (US); Harry A. Gray, JR., Cleveland Heights, OH (US); Joanne M. Stack-Oyen, Chesterland, OH (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/376,475

(22) PCT Filed: Jul. 23, 2007
(86) PCT No.: PCT/US07/74096

§ 371 Date: Feb. 5, 2009

Related U.S. Application Data
(60) Provisional application No. 60/821,834, filed on Aug. 9, 2006.

(51) Int. Cl. A61B 6/03; A61B 6/08

(52) U.S. Cl. 378/20; 378/4

(57) ABSTRACT

A computed tomography system (100) includes a stationary gantry (104) that houses at least one x-ray source (112) that rotates about an examining region (120) and at least one detector (116) that resides opposite the examining region (120) from the at least one x-ray source (112). The stationary gantry (104) further includes an annular ring (132) disposed about the examination region (120) in a path of the x-ray beam between the at least one x-ray source (112) and the at least one detector (116), wherein the annular ring (132) is substantially opaque to visible light.

The present application relates to medical imaging systems. It finds particular application to computed tomography (CT) and, more particularly to reducing the ability of a patient being scanned from viewing components within the gantry.

A typical CT system used for medical imaging includes a housing generally referred to as a gantry. The gantry houses many of the physical components used for generating and detecting x-rays, including x-ray tubes and detectors. The gantry has also included a generally annular housing portion which defines an examination region for receiving a subject such as a human patient.

When scanning a subject with such a system, an x-ray tube is activated to generate and emit x-rays that traverse an imaging region in which the subject is variously positioned. While traversing the subject, a number of the x-rays are attenuated by the subject in proportion to the density thereof. Some of the x-rays interact with matter in a manner which causes the x-rays to lose energy and change direction (e.g., Compton scattering). The attenuated radiation is detected by the detector and is used to reconstruct tomographic data representative of the scanned subject. Since matter in the path of the x-ray beam can attenuate, scatter or otherwise affect the characteristics of the x-rays, objects (e.g., filters, etc.) intentionally placed within the path are often selected according to their effect on the x-ray characteristics.

Governmental regulations have also required that CT systems provide the user with a visual indication of the location of a tomographic image plane or otherwise of a known reference plane. Where a light source is used to indicate the location of the reference plane, the light source must permit visual identification of the reference plane under ambient light conditions of up to 500 lux. See Performance Standards for Ionizing Radiation Emitting Products 21 C.F.R. 1020.33(g); A Guide for the Submission of Initial Reports on Computed Tomography X-ray Systems, U.S. Food and Drug Administration, December 1985. To satisfy this requirement, an alignment laser has been located inside the gantry. The alignment laser has been implemented as a red laser which provides a visual indication of the location of the x-ray beam on the patient or otherwise in the examination region.

Consequently, the inner housing portion of conventional CT systems has included a substantially x-ray and optically transparent ring member disposed in the path of the x-ray and alignment laser beams. More specifically, the ring member has been located between the x-ray source and the imaging region and the one or more detectors and the imaging region. In addition to facilitating the use of a material having the requisite x-ray characteristics, the ring has provided an optically transparent path for the alignment laser beam. In one system, the ring has been fabricated from clear Lexan® 9034 polycarbonate material having a thickness of approximately 0.080 inches (2.03 mm). The Lexan 9034 material, which is manufactured by General Electric Company of Fairfield, Conn., USA, has a visible light transmission of approximately eighty eight percent (88%).

One trend in CT systems has been the widespread adoption of multi-slice systems, which provide a greater longitudinal or z-axis coverage and thus a range of clinical benefits compared to conventional single slice systems. As the number of slices and hence the longitudinal coverage of multi-slice systems has increased, so has the longitudinal extent of the x-ray beam and the detector. Thus, longitudinal extent or width of the ring has been increased accordingly. In one sixty four (64) slice system, the ring has had a width of approximately 3.875 inches (9.824 cm).

Unfortunately, however, the patient is often positioned in proximity to the ring. As a result, increasing the width of the ring has rendered more and more of the internal structure and components of the system visible to the patient. This can be especially problematic in systems in which the system includes rotating or other moving components which are visible to the patient through the ring. To provide an improved patient experience, it is thus desirable to limit the patient's awareness of the internal components of the gantry in a manner consistent with the imaging and other functional requirements of the CT system.

Aspects of the present application address the above-referenced matters and others.

According to one aspect, an x-ray computed tomography apparatus includes a gantry and an x-ray source disposed in the gantry and which generates an x-ray beam which traverses an examination region. The apparatus also includes an x-ray sensitive detector disposed in the gantry opposite the examination region from the x-ray source, and a generally annular ring disposed about the examination region in a path of the x-ray beam and between the x-ray source and the detector. The ring is substantially opaque to visible light.

According to another aspect, an apparatus includes an ionizing radiation source which rotates about an examination region and a radiation sensitive detector. The detector receives radiation generated by the radiation source which radiation has traversed the examination region. The apparatus also includes a generally annular member disposed in a path of the radiation between the radiation source and the examination region and between the examination region and the detector. The member includes a visible light transmission characteristic which renders the detector substantially invisible to a human patient disposed in the examination region.

According to another aspect, a computed tomography apparatus includes a gantry and an ionizing radiation source disposed in an interior of the gantry. The radiation source generates ionization radiation at a plurality of angular positions with respect to the examination region. The apparatus also includes a radiation sensitive detector disposed in the interior of the gantry and which receives radiation generated by the radiation source. The apparatus also includes a generally annular gantry portion operatively connected to the gantry and which faces the examination region. At least a portion of the annular gantry portion disposed in a path of the ionizing radiation is substantially transmissive of the ionizing radiation and substantially opaque to visible light.

The invention may take form in various components and arrangements of components, and in various steps and arrangements of steps. The drawings are only for purposes of illustrating the preferred embodiments and are not to be construed as limiting the invention.

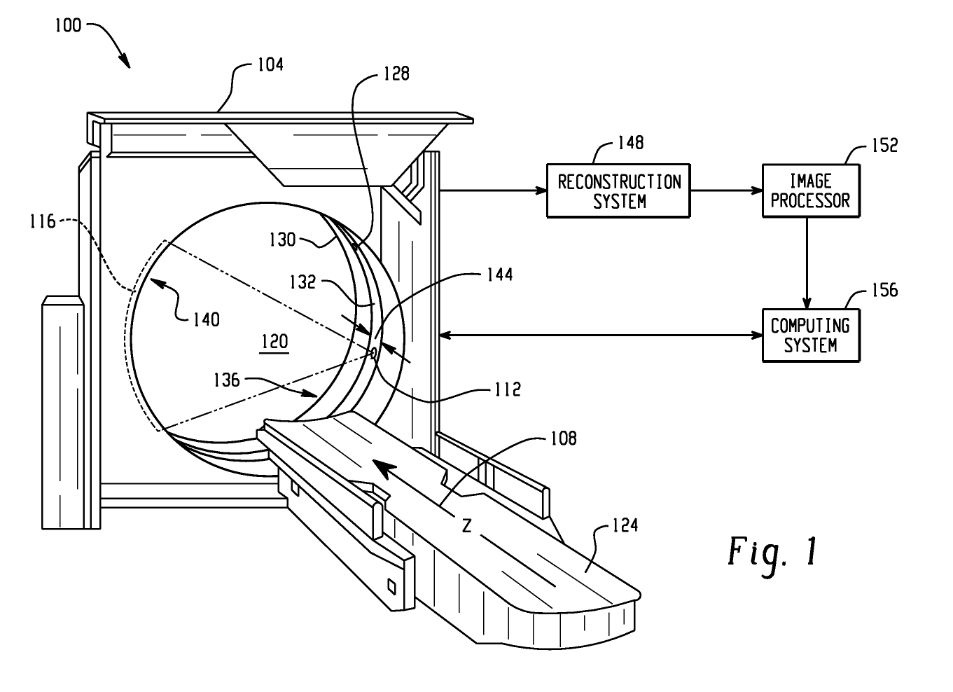

FIG. 1 illustrates an exemplary medical imaging system having an x-ray transmissive ring that is substantially opaque to visible light.

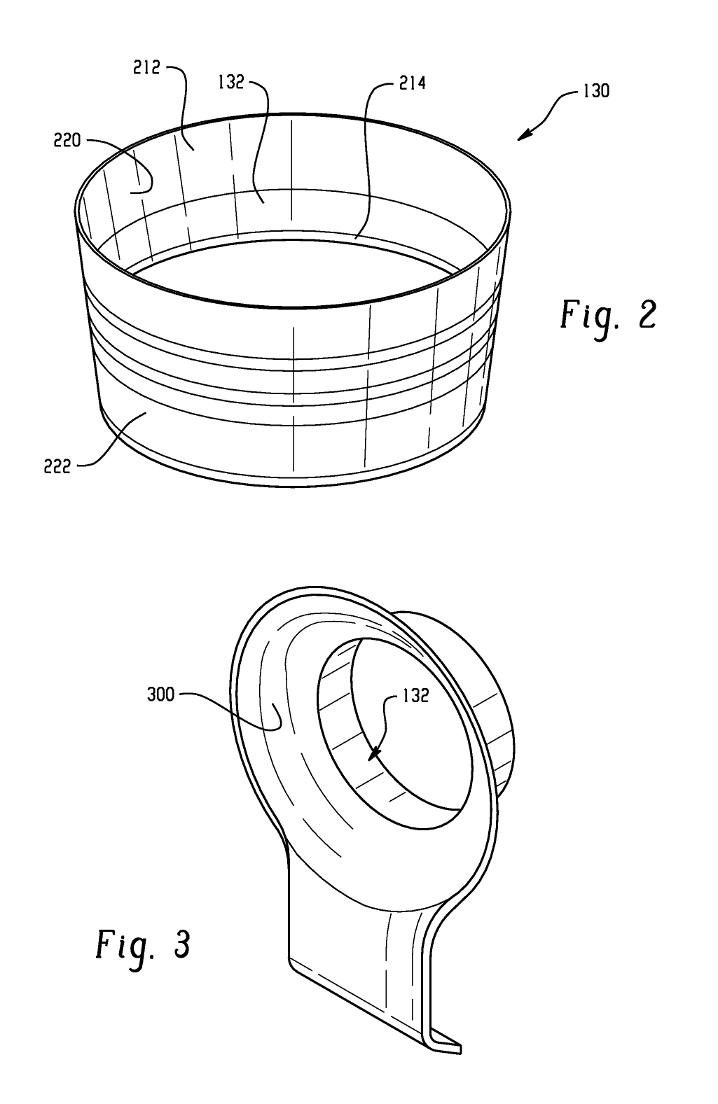

FIG. 2 illustrates an exemplary embodiment of an annular housing portion including the x-ray transmissive ring.

FIG. 3 illustrates an embodiment in which the x-ray transmissive ring is attached to a cone portion.

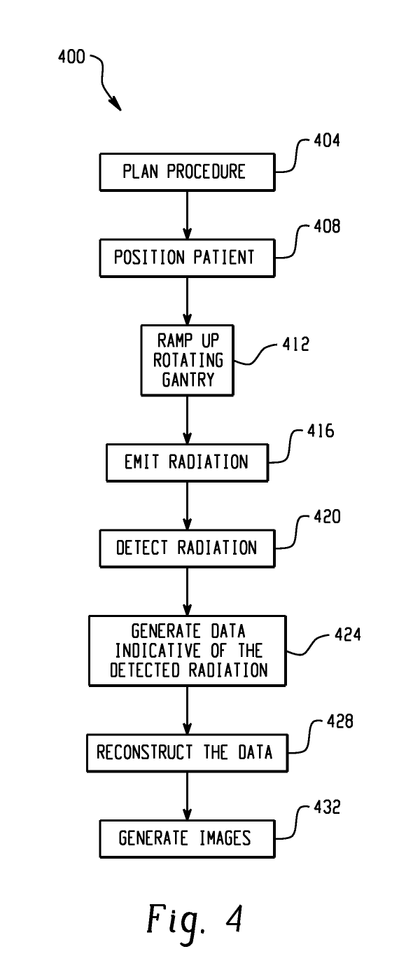

FIG. 4 illustrates an imaging method.

With reference to FIG. 1, a medical imaging system 100 includes a generally stationary housing or gantry 104. In the illustrated third generation CT system, an inner or rotating portion (not visible) is disposed in an interior of the gantry 104. The rotating portion, which rotates about a z-axis 108, supports an x-ray source 112 such as an x-ray tube that generates a generally conical or fan shaped radiation beam.

The rotating portion also supports an x-ray sensitive detector 116 that subtends an angular arc on an opposite side of an examination region 120. The detector 116 is a multi-slice detector that includes multiple rows or slices of detector elements that extend in the z-axis direction and multiple columns of detector elements that extend in a traverse direction. In one instance, the detector 116 includes sixty-four (64) or more such slices. The rotating portion also supports a high voltage generator, a collimator, an anti-scatter grid, and/or other components relevant to the operation of the system.

During imaging, the rotating portion and the components disposed thereon rotate about the examination region 120 so as to acquire projection data at a plurality of angular positions with respect thereto. As temporal resolution and data acquisition time are a function of the rotating portion rotation rate, rotation rates are typically on the order of about two rotations per second or greater, with the maximum rotation rate typically being limited by factors such as the available tube power, required radiation flux, mechanical capabilities of the rotating portion, and the like. Relatively slower rotation speeds are also contemplated.

A patient support 124 such as a couch supports a patient in the examination region 120. The patient support 124 is movable along the z-axis 108 in coordination with the rotation of the x-ray source 112 to facilitate helical, axial, or other desired scanning trajectories.

Also disposed in an interior of the gantry 104 is an alignment light source 128 such as a 1.0 milliwatt (mW) red laser. The light source 128 generates a light beam that is directed into the examination region 120 and is visible on the patient support 124 and/or the patient. The light beam, which has a known physical relationship to the x-ray beam, is used to facilitate positioning the patient in the examination region 120 in connection with a scan. More particularly, the beam permits the identification of a reference plane under ambient light conditions of at least 500 lux.

The gantry 104 further includes a first generally annular housing portion 130 that faces the examining region 120. The housing 130 includes a generally annular ring 132 that is disposed about the examination region 120 in the path of the x-ray and alignment light beams. The ring 132 is positioned relative to the x-ray source 112 so that x-rays generated by the source 112 pass through the ring 132 on a first side 136 of the examination region 120, traverse the examination region 120, again pass through the ring 132 on an opposite second side 140 of the examination region 120, and reach the detector 116. Light generated by the alignment light source 128 likewise transits the ring 132 prior to illuminating the patient support 124 and/or the patient.

The ring 132 has a longitudinal dimension or width 144 that is at least as wide as the x-ray beam at the location of the ring 132. Hence, x-rays that traverse the examination region 120 and are received by the detector 116 pass through the ring 132. In the illustrated sixty-four (64) slice system, the ring has a radius of about 27.559 degrees, a width of about 3.875 inches (9.842 cm), and a thickness of about 0.080 inches (2.032 cm).

In the illustrated embodiment, the ring 132 performs a variety of functions. In addition to providing a suitable path for x-rays and the alignment light beam, the ring 132 also serves as a mechanical barrier between the examination region 120 and the interior of the gantry 104. For example, the ring 132 prevents an operator or patient from inadvertently contacting the interior of the gantry 104. The ring 132 also protects the interior components from bodily fluids and other contaminants that are sometimes present during a scan.

As will be described in greater detail below, the ring 132 also reduces the ability of the patient or other individuals to see the rotating portion and/or other interior components of the gantry 104. For example, the ring 132 is transmissive to x-ray radiation and substantially opaque to visible light. This enables x-rays and a suitable amount of laser light to pass through the ring 132, while generally preventing a patient or other individual from seeing the interior components of the gantry 104.

A reconstructor 148 reconstructs projection data from the detectors to generate volumetric data indicative of the interior anatomy of the patient. An image processor 152 processes the volumetric image data generated by the reconstructor 148 for display in human readable form.

A general purpose computing system serves as an operator console 156. The operator console 156 includes human readable output devices such as a monitor and/or printer and input devices such as a keyboard and/or mouse. Software resident on the console 156 allows the operator to control the operation of the system 100 by establishing desired scan protocols, initiating and terminating scans, viewing and otherwise manipulating the volumetric image data, and otherwise interacting with the system 100.

The ring 132 will now be described in further detail. FIG. 2 illustrates one implementation in which the ring 132 forms an integral part of the annular housing portion 130. In this implementation, the housing portion 130 is fabricated as a unitary structure having a width that is greater than the required width 144 of the ring 132. The housing portion 130 is suitably attached to a portion of the gantry 104 facing the examining region 120 or otherwise to relatively more front and rearward portions of the gantry 104.

The annular housing portion 130 is fabricated from a polycarbonate material that is impregnated, treated, or tinted so as to render it substantially optically opaque. In one non-limiting implementation, the material has a grey, black, or smoked appearance and an average visible light transmission of about seventeen percent 17%. A particular advantage of such an implementation it renders the interior components of the gantry 104 substantially invisible to the patient, while still providing a suitable path for the alignment light beam.

One suitable material for the housing portion 130 is impregnated Tuffak® polycarbonate material, which is produced by Atoglas International, Arkema Inc., Philadelphia, Pa., USA. The optical characteristics of this material also pass laser light such that the laser beam generally is not diffused. As a result, laser light passing through the material can be used to position the patient support 124 or the patient with respect to the x-ray source 112. The material typically comes from the manufacturer with an average visible light transmission tolerance of plus or minus about four percent (+−4%). The material also has x-ray transmissive characteristics similar to those of the Lexan 9034 material.

With continuing reference to FIG. 2, awareness of the gantry 104 interior components may optionally be further reduced by coating or otherwise covering one or more longitudinally extending portions 212, 214 of the housing portion 130. This can be accomplished by painting, silk screening, or otherwise covering the desired portions 212, 214 of the annular housing portion 130. A layer of a suitable material such as a still more optically opaque polymer or polymer film may also be applied.

Where the covering is applied on a side 220 of the housing portion 130 which faces the examination region 120, the covering may be selected to provide a color or other visual appearance which complements the remainder of the gantry 104. Alternately or additionally, the covering may also be applied to the side 222 of the housing 130 which faces the interior of the gantry 104. To avoid the need for characterizing the x-ray and light transmission characteristics material, the covering is advantageously not applied at the location of the ring 132.

In one implementation, the housing portion 130 is fabricated as a generally flat structure such as polycarbonate sheet having the desired optical characteristics. The structure is subsequently processed to form a closed ring by rolling the material and bonding the ends together. The housing 130 is then affixed to the gantry 104 as desired.

FIG. 3 illustrates an implementation in which the ring 132 is substantially permanently physically attached to a front cover or cone 300 so that the ring 132 and cone 300 move together as a unitary assembly. Such an implementation is particularly advantageous where the front cone 300 is pivotally or otherwise movably mounted to the gantry 104 so as to provide access to the gantry 104 interior, for example for servicing. In an alternative implementation, the ring 132 is may be attached to a rear or back cone (not shown).

In another implementation, the ring 132 is formed as a separate structure of a desired width and thickness. The ring 132 is mounted at a desired position in the path of the x-ray and light beams, for example by suitably attaching the ring to the housing portion 130, the front cone 300, the back cone, or otherwise to a desired portion of the gantry 104. For example, in one instance the ring 132 is mechanically attached to the housing portion 130. In another instances, the ring 132 is chemically attached to the housing portion 130. Other attachment techniques are also contemplated herein.

As still another alternative, the housing portion 130 and/or the ring 132 may be fabricated as two more pieces each having a desired circumferential or longitudinal extent. The housing 130 and/or the ring 132 may also be molded or otherwise formed in the desired shape.

Although the above discussion has focused on the use of an impregnated polycarbonate, other polymeric and non-polymeric materials having desirable x-ray and optical characteristics are contemplated herein. Examples of suitable materials include polyester, mylar, co-polymer, thermoplastic, polyethylene, polypropylene, PVC, acrylic, and the like.

In another embodiment, the ring 132 is formed from multiple layers. In this embodiment, a first transparent polycarbonate or other layer having desirable x-ray transmissive characteristics is joined with a second layer having known x-ray characteristics and desirable optical characteristics. The two layers can be joined by laminating, spraying, silk screening, painting, etc. the second layer over the first layer.

As the visibility of the interior of the gantry 104 is a function of factors such as the ambient lighting conditions, the interior lighting of the gantry 104, the width 144 of the ring 132, the characteristics of the interior components of the gantry 104, the proximity of the patient and/or the interior components to the ring 132, and the anticipated sensitivity of the patients, the optical transmissivity of the ring 132 may be established at a value which is other than seventeen percent (17%), with a tolerance greater or smaller than plus or minus four percent (+−4%).

For example, the present inventors have observed that the gantry 104 interior components tend to be relatively less visible under relatively brighter external ambient lighting conditions. Consequently, a relatively more optically transmissive (or stated conversely, a relatively more optically opaque) material may be used where the system 100 is expected to be operated under relatively brighter lighting conditions. Stated conversely, the interior components will be relatively less visible for a material having a given transmissivity. As increasing the width 144 of the ring 132 tends to increase the visibility of the interior components, systems having a relatively wider ring 132 may require a relatively lower optical transmissivity.

In the system described in connection with FIG. 1, a 1.0 mW red alignment light source 128 was used. However, it is to be understood that other mono or polychromatic lasers with different power ratings can be used. For instance, a green or other color alignment light source 128 with less or greater power can alternatively be used. The alignment light source 128 may also be omitted, in which case the ring 132 may be fabricated from a material which is or is otherwise processed to be relatively diffuse to visible light and still maintains suitable x-ray attenuation characteristics.

It is also to be appreciated that the ring 132 can be used with fourth (4^{th}) and other generation systems using single or multi-slice detectors. In addition, the above is described in connection with a CT imaging system. However, it is to be appreciated that other imaging modalities, including, but not limited to, nuclear imaging are also contemplated herein.

Operation of the imaging system 100 will now be described in relation to FIG. 4. At 404, the operator interacts with the console 156 to plan the procedure. Such interaction includes selecting imaging protocols and the like.

At 408, the operator optionally uses the internal alignment light 132 to position the patient within the examining region 120, for example by moving the patient support 124 until the light beam is located at a desired position relative to the patient and/or the patient support.

At 412, scanning begins with the rotating portion ramping up to an appropriate rotational speed. This assumes an axial or spiral scan is being performed. In instances in which a pilot scan, a scout scan, or the like is performed, the rotating portion remains in a static position, although it may have to rotate to a suitable angular position before such scanning.

At 416, the x-ray source 112 generates and emits radiation. As described above, such radiation transits the ring 132 and traverses in the examining region 120.

At 420, x-rays that traverse the examining region 120, transit another portion of the ring 120, and illuminate the detector 116.

At 424, the detector 116 generates data indicative of the detected radiation.

At 428, the data is reconstructed by a reconstructor 148 that generates volumetric image data therefrom.

At 432, the image processor 152 generates one or more images from the reconstructed volumetric data.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. An x-ray computed tomography apparatus comprising:
a gantry;
an x-ray source disposed in the gantry and which generates an x-ray beam which traverses an examination region;
an x-ray sensitive detector disposed in the gantry opposite the examination region from the x-ray source;
a generally annular ring disposed about the examination region in a path of the x-ray beam and between the x-ray source and the detector, wherein the ring is substantially opaque to visible light.

2. The apparatus of claim 1 wherein the ring has a visible light transmission of about seventeen percent.

3. The apparatus of claim 1 wherein the ring is fabricated from an impregnated polycarbonate.

4. (canceled)

5. The apparatus of claim 1 wherein the ring includes a first material layer and a second material layer, and wherein the second material layer is substantially optically opaque.

6. (canceled)

7. The apparatus of claim 1 including
an alignment light source disposed in the gantry and which generates an alignment light for positioning an object in the examination region, and wherein the ring is disposed in the path of the light beam.

8. The apparatus of claim 7 wherein the light source includes a laser and wherein the alignment light provides a visible identification of a reference plane under ambient light conditions of greater than or equal to 500 lux.

9. The apparatus of claim 1 wherein the ring has a width which is greater than or equal to a width of the x-ray beam at a location of the ring.

10. (canceled)

11. The apparatus of claim 1 wherein the ring renders the x-ray source and the x-ray detector substantially invisible to a human patient disposed in the examination region.

12. The apparatus of claim 1 wherein the gantry includes a generally annular housing portion disposed about the examination region, and wherein the ring is attached to the housing portion.

13. The apparatus of claim 1 wherein the gantry includes a cover which is movably attached to the gantry so as to provide access to an interior portion thereof, and wherein the ring is attached to the cover for movement therewith.

14. The apparatus of claim 1 wherein the gantry includes a generally annular housing portion disposed about the examination region, and wherein the ring forms an integral part of the housing portion.

15. An apparatus comprising:
an ionizing radiation source which rotates about an examination region;
a radiation sensitive detector which receives radiation generated by the radiation source, which radiation has traversed the examination region;
a generally annular member disposed in a path of the radiation between the radiation source and the examination region and between the examination region and the detector, wherein the member includes a visible light transmission characteristic which renders the detector substantially invisible to a human patient disposed in the examination region.

16. The apparatus of claim 15 wherein the ring has a visible light transmission of about seventeen percent.

17. The apparatus of claim 15 wherein the apparatus includes
a patient support which supports a human patient in the examination region;
a light source which generates visible light,
wherein the visible light passes through the member and is visible on at least one of the patient support and a patient disposed on the patient support.

18. (canceled)

19. (canceled)

20. The apparatus of claim 15 wherein the ionizing radiation source is an x-ray source and the detector is a multi-slice computed tomography detector.

21. The apparatus of claim 15 including
a gantry;
a generally annular housing portion operatively connected to the gantry and which faces the examination region, wherein the member forms at least a part of the generally annular housing portion.

22. The apparatus of claim 15 including a cover which is movably attached to the gantry so as to provide access to an interior thereof, and wherein the member is attached to the cover for movement therewith.

23. (canceled)

24. (canceled)

25. (canceled)

26. A computed tomography apparatus comprising:
a gantry;
an ionizing radiation source disposed in an interior of the gantry and which generates ionization radiation at a plurality of angular positions with respect to the examination region;
a radiation sensitive detector disposed in the interior of the gantry and which receives radiation generated by the radiation source, which radiation has traversed the examination region;
a generally annular gantry portion operatively connected to the gantry and which faces the examination region, wherein at least a portion of the annular gantry portion disposed in a path of the ionizing radiation is substantially transmissive of the ionizing radiation and substantially opaque to visible light.

27. (canceled)

28. The apparatus of claim 26 wherein the polymer is a tinted polymer, and wherein the tinting renders the polymer substantially opaque to visible light.

29. The apparatus of claim 26 wherein the at least a portion of the annular gantry portion includes a first material layer and a second material layer, and wherein the second material layer is substantially opaque to visible light.

30. The apparatus of claim 29 wherein the second material layer includes one of a surface coating and a film.

31. The apparatus of claim 26 wherein the at least a portion of the annular gantry portion has a visible light transmissivity of about seventeen percent.

32. The apparatus of claim 26 including an alignment laser disposed in an interior of the gantry, wherein the at least a portion of the annular gantry portion is disposed in a path of the light beam, and wherein the light beam provides a visual indication of a reference plane under ambient lighting conditions of at least 500 lux.

33. (canceled)
